(19) United States
(12) Patent Application Publication
(10) Pub. No.: US 2010/0168719 A1
(43) Pub. Date: Jul. 1, 2010

(54) PROCESSING SYSTEM AND COLLECTING DEVICE THEREOF

(75) Inventor: Tuan-Min Chen, Hsinchu City (TW)

Correspondence Address: QUINTERO LAW OFFICE, PC, 615 Hampton Dr, Suite A202, Venice, CA, 90291, US

(73) Assignee: INDUSTRIAL TECHNOLOGY RESEARCH INSTITUTE, HSINCHU (TW)

(21) Appl. No.: 12/464,771

(22) Filed: May 12, 2009

(30) Foreign Application Priority Data
Dec. 29, 2008 (TW) TW97151212

(51) Int. Cl. A61M 27/00

(52) U.S. Cl. 604/543; 604/378

(57) ABSTRACT

A processing system is utilized to process and absorb a fluid discharging from a source. The processing system includes an absorbing device to absorb the fluid, a driving device and a collecting device. The driving device connected to the absorbing device drives the fluid collected by the absorbing device to move, and the collecting device connected to the driving device is utilized to collect the fluid driven by the driving device. The collecting device includes a base and an absorbing element. The based includes a first layer, a second layer and a guiding area formed by the first and second layers. The guiding area includes two channels connected to each other. The absorbing element enclosed by the first and second layers of the base is disposed next to the guiding area, so that the fluid guided by the two channels of the guiding area is absorbed by the absorbing element.

### CROSS REFERENCE TO RELATED APPLICATIONS

This Application claims priority of Taiwan Patent Application No. 97151212, filed on Dec. 29, 2008, the entirety of which is incorporated by reference herein.

### BACKGROUND OF THE INVENTION

1. Field of the Invention

The present invention relates to a collecting device, and in particular relates to a processing system and a collecting device thereof capable of absorbing and collecting waste liquid or blood from a wound.

2. Description of the Related Art

Negative Pressure Wound Therapy (NPWT) is an auxiliary physical therapy method. Due to effective performance thereof, the NPWT is widely applied to treat large-sized and deep wounds for cleaning acrid discharging from wounds, providing a moist theoretic environment, protecting the wound and decreasing wound recovery duration. In the process of the NPWT, after the dressed wound is hermetically wrapped, a conduit is connected between the dressed wound and a negative pressure pump, thereby inducting negative pressure inside of the wrapped wound. A rigid negative pressure collecting device disposed between the wrapped wound and the negative pressure pump is utilized to collect waste liquid drained from the wound, so that the waste liquid can be prevented from entering the negative pressure pump.

Further, in the process of the NPWT, a negative inlet connected to the dressed wound is left, and negative pressure is transmitted to the wound by the pipes and an electric negative pressure pump. A negative collecting device configured between the pipes and the negative pressure pump is utilized to absorb the waste liquid from the wound and prevent the waste liquid from entering the negative pressure pump. Due to the negative pressure environment, the waste liquid collecting device is a container generally made of rigid and transparent plastic material and capable of withstanding negative pressure. Some of the containers are stored with absorbing resin therein, and some must be uprightly placed for preserving the collected liquid therein.

FIG. 1 is a schematic block diagram of a conventional negative pressure wound therapy (NPWT) N. The NPWT N comprises a dressing assembly (not shown) disposed on a wound Q, a waste liquid collecting device K2 and a (negative pressure) pump K3. With the configuration of the dressing assembly, the waste liquid collecting device K2 and the pump K3, a negative region K1 can be formed to absorb a waste liquid W from the wound Q. Basically, the waste liquid W is not allowed to enter the pump K3, and the waste liquid collecting device K2 configured between the wound Q and the pump K3 is utilized to absorb the waste liquid W from the wound Q and to stop the waste liquid W from entering the pump K3. The waste liquid collecting device K2 is a container made of rigid and transparent plastic material and capable of withstanding negative pressure. To avoid waste liquid W from entering back into the pump K3 while passing through the waste liquid collecting device K2, negative pipes are configured at the front of the waste liquid collecting device K2, and the NPWT N is uprightly disposed to prevent any pollutant from entering the pump K3, i.e., the NPWT N cannot be slantedly placed. Thus, it is inconvenient to carry the NPWT N.

For example, U.S. Pat. No. 6,056,730A1 discloses a collecting device provided with a disposable liquid-collecting bag operated under negative pressure. A rigid outer container is utilized to sustain the liquid-collecting bag so that the liquid-collecting bag is prevented from being compressed by atmospheric pressure. The design of the sealed connection of the storing bag, supporting container, waste-liquid conduit and pump has been disclosed.

### BRIEF SUMMARY OF THE INVENTION

In view of the inconveniences and problems described above, the invention provides a portable negative pressure wound therapy facility. In the NPWT, a small-sized and pollutant-separable pump is provided and a positive terminal thereof is connected to a collecting apparatus. Incorporated with modern material and manufacturing technologies, the collecting device is characterized with features such as being soft and well-fitting, being light and portable, having low resistance from the collected liquid, being observable for measuring the collected liquid, and being adequate for operational requirements of new facilities.

The collecting device of the invention is utilized to collect a fluid. The collecting device comprises a base, a first guiding element and an absorbing element.

In one embodiment, the invention further provides another collecting device utilized to collect a fluid. The collecting device comprises a base and an absorbing element. The base comprises a first layer, a second layer, and a guiding region formed by the first and second layers and comprising a first channel and a second channel connected to the first channel. The absorbing element enclosed by the first and second layers of the base is disposed next to the guiding region of the base to absorb the fluid guided by the first and second channels of the guiding region of the base.

In another embodiment, the invention further provides a processing system utilized to process and absorb a fluid discharging from a source. The processing system comprises an absorbing device and a collecting device. The absorbing device absorbs the fluid discharging from the source. The driving device connected to the absorbing device is utilized to move the fluid collected by the absorbing device. The collecting device connected to the driving device and utilized to collect a fluid comprises a base and an absorbing element. The base comprises a first layer, a second layer, and a guiding region formed by the first and second layers and comprising a first channel and a second channel connected to the first channel. The absorbing element enclosed by the first and second layers of the base is disposed next to the guiding region of the base to absorb the fluid guided by the first and second channels of the guiding region of the base.

### BRIEF DESCRIPTION OF THE DRAWINGS

The present invention can be more fully understood by reading the subsequent detailed description and examples with references made to the accompanying drawings, wherein:

FIG. 1 is a schematic block diagram of a conventional negative pressure wound therapy (NPWT);

FIG. 2 is a schematic block diagram of a processing system of the invention;

FIG. 3 is a schematic view of a collecting device of a first embodiment of the invention;

FIG. 4A is a sectional view showing the inner structure of the collecting device along line (s11-s11) in FIG. 3;

FIG. 4B is a schematic view of the collecting device while collecting the fluid in FIG. 4A;

FIG. 5A shows another embodiment of the inner structure of the collecting device of FIG. 3;

FIG. 5B is a schematic view showing the embodiment while collecting the fluid in FIG. 5A;

FIG. 6 is a schematic view of a collecting device of a second embodiment of the invention;

FIG. 7A is a sectional view showing the inner structure of the collecting device along line (s21-s21) in FIG. 6;

FIG. 7B is a sectional view showing the inner structure of the collecting device along line (s22-s22) in FIG. 6;

FIGS. 8A and 8B show another embodiment of the inner structures of the collecting device of FIGS. 7A and 7B, respectively;

FIG. 9A is a schematic view of a collecting device of a third embodiment of the invention;

FIG. 9B is a schematic view of the collecting device while collecting the fluid in FIG. 9A;

FIG. 10A is a schematic view of a collecting device of a fourth embodiment of the invention; and

FIG. 10B is a schematic view of the collecting device while collecting the fluid in FIG. 10A.

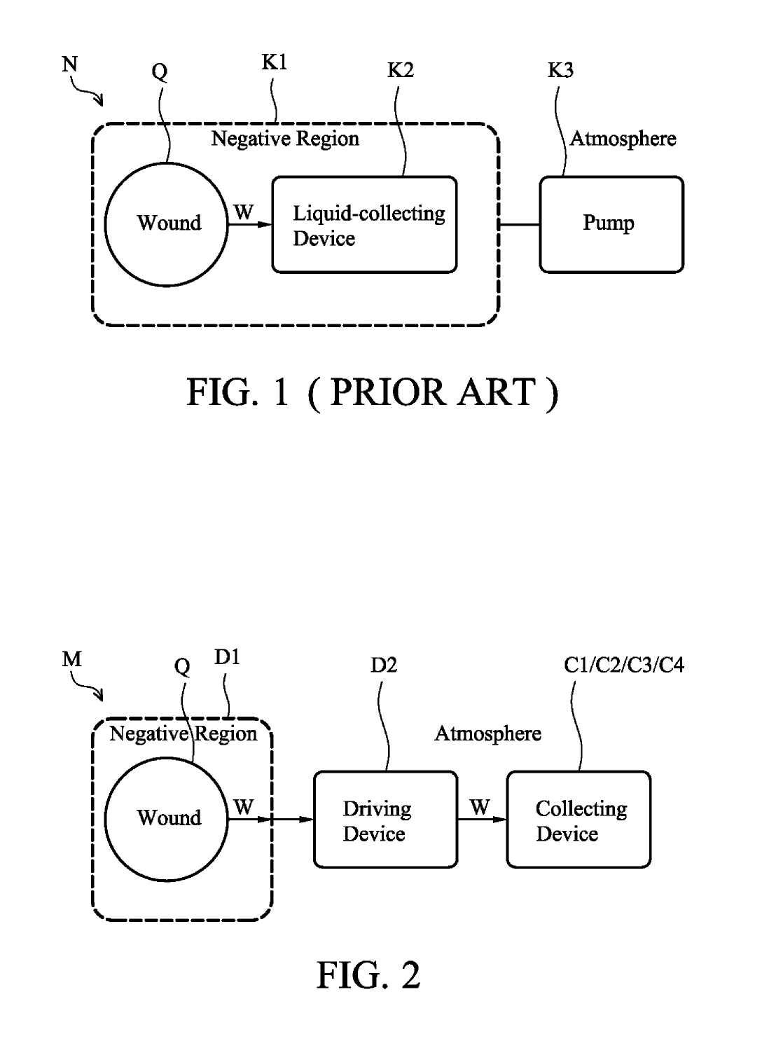

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

FIG. 2 is a schematic block diagram of a processing system M. In an atmospheric environment (one atmospheric pressure), the processing system M is utilized to process and absorb a fluid W (e.g., waste liquid or blood) discharging from a source Q (e.g., wound). In the following description, the fluid W is the waste liquid W and the source Q is the wound Q.

The processing system M comprises an absorbing device D1, a driving device D2 and collecting devices C1/C2/C3/C4. The absorbing device D1 is utilized to absorb the waste liquid W discharging from the source Q located in a negative pressure region. In this embodiment, the absorbing device D1 comprises a seam patch, a flexible sucker and biologically compatible porous material (not shown). The driving device D2 connected to the absorbing device D1 is separably or detachably disposed between the absorbing device D1 and the collecting device C1/C2/C3/C4. The waste liquid W collected by the absorbing device D1 is moved to the driving device D2, and then the waste liquid W is moved to the collecting device C1/C2/C3/C4 from the driving device D2. That is, the negative region is formed around the wound Q by the absorbing device D1 and the driving device D2, thereby absorbing the waste liquid W discharging from the wound Q. In this embodiment, the driving device D2 is a negative pressure pump. In the following description, the driving device D2 is the negative pressure pump D2.

A detailed description is given in the following collecting device C1, C2, C3 and C4 with reference to the accompanying drawings.

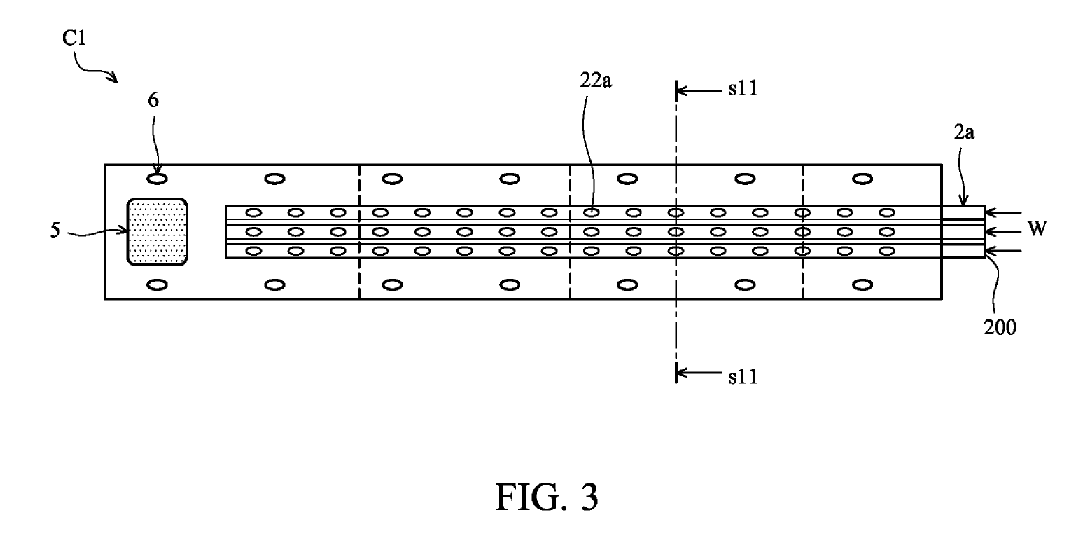

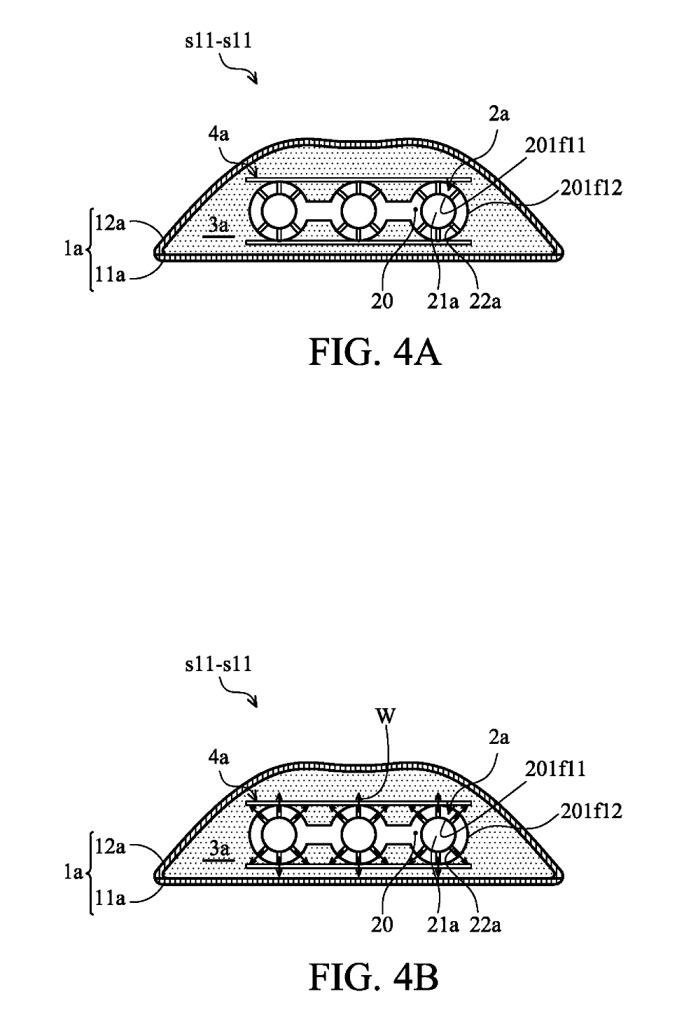

FIG. 3 is a schematic view of a collecting device C1 of a first embodiment, FIG. 4A is a sectional view showing the inner structure of the collecting device C1 along line s11-s11 in FIG. 3, and FIG. 4B is a schematic view of the collecting device C1 while collecting the waste liquid W in FIG. 4A.

In FIGS. 3 and 4A, the collecting device C1 comprises a base 1a, at least a first guiding element 2a, an absorbing element 3a, a second guiding element 4a, an air permeable region 5 and a display region 6.

The base 1a is a hollow membrane structure comprising a first layer 11a and a second layer 12a. In this embodiment, the first and second layers 11a and 12a of the base la are formed by a rectangular or strip-like water-resist separation layer or a water-resist plastic membrane, so that the base la can suitably conform to any part of the human body and secure to the skin thereon.

The first guiding element 2a is a longitudinal hollow or pipe structure disposed between the first and second layers 11a and 12a of the base 1a, wherein the first guiding element 2a is completely enclosed by the first and second layers 11a and 12a of the base 1a. The first guiding element 2a comprises a hollow body 20 having an inner surface 201f11 and an outer surface 201f12. The inner surface 201f11 forms a first channel 21a, and a plurality of second channels 22a formed on the hollow body 20 is connected between the inner and outer surfaces 201f11 and 201f12. The outer surface 201f12 disposed next to the absorbing element 3a is connected to the inner surface 201f11 via the second channels 22a. In this embodiment, the first guiding element 2a is made of silicone, and the amount of the first guiding element 2a is three, wherein the first guiding element 2a is longitudinally and adjacently arranged. In other embodiments, the guiding element can be made of rubber or plastic material.

The absorbing element 3a is disposed between the base 1a and the first guiding elements 2a, and the first guiding elements 2a enclosed by the absorbing element 3a is permanently enclosed between the first and second layers 11a and 12a of the base 1a. In this embodiment, the absorbing element 3a is made of particulate water-absorbing resin material such as starch superabsorbent polymer, sodium polyacrylate and potassium polyacrylate. In other embodiments, the absorbing element can be made of hydrophilic plastic foam or water-absorbing foam material.

The second guiding element 4a is a transparent water-guiding layer, which is disposed between the second channels 22a of the first guiding element 2a and the absorbing element 3a, or covers the second channels 22a of the first guiding element 2a. With the arrangement of the second guiding element 4a, the waste liquid W flowing from the second channels 22a of the first guiding element 2a is allowed to enter the absorbing element 3a, and the particulate absorbing element 3a is not allowed to enter the first channel 21a via the second channels 22a.

The air permeable region 5 is disposed on the first and second layers 1a and 12a of the base 1a to connect the interior of the base 1a to the exterior thereof, expelling air containing in the base 1a, the guiding element 2a and the absorbing element 3a to the exterior when the waste liquid W travels in the collecting device C1. In this embodiment, the air permeable region 5 comprises at least one vent hole. Note that water and breathable membrane and activated carbon or other composite substances capable of eliminating odor are installed on the vent hole(s), thereby sealing the vent hole(s), preventing the release of the waste liquid W and expelling the air without odor.

The display region 6 comprises wet-display dots disposed on the first and second layers 11a and 12a of the base 1a and connect to the absorbing element 3a to display the condition e.g., saturated state of the absorbing element 3a, containing the waste liquid W. In this embodiment, the display region 6 made of water-discoloration material can be formed on the inner surface of the first and second layers 11a and 12a of the base 1a by printing.

When the waste liquid W driven by the negative pressure pump D2 is transmitted to the collecting devices C1 through an inlet 200 (shown in FIG. 3), the waste liquid W traveling through the first and second channel 21a and 22a of the first guiding element 2a and the second guiding element 4a is absorbed by the absorbing element 3a. Further, the air contained in the base 1a is adequately expelled to the exterior via the air permeable region 5, and the display region 6 shows the corresponding region of the absorbing element 3a being saturated or not by the change of the color thereof. Therefore, the absorption rate can be statistically estimated by the amount of the colored absorbing element 3a.

Further, because the first guiding element 2a is a longitudinal hollow structure disposed between the first and second layers 11a and 12a of the base 1a, i.e., the first guiding element 2a penetrates the base 1a, the waste liquid W transmitted by the negative pressure pump D2 rapidly and uniformly flows to the unsaturated regions of the absorbing element 3a via the first and second channel 21a and 22a of the first guiding element 2a. When some of the second channels 22a impede the discharge resistance from the waste liquid W, the waste liquid W can be swiftly discharged from other second channels 22a, thereby alleviating the discharge resistance of the negative pressure pump D2.

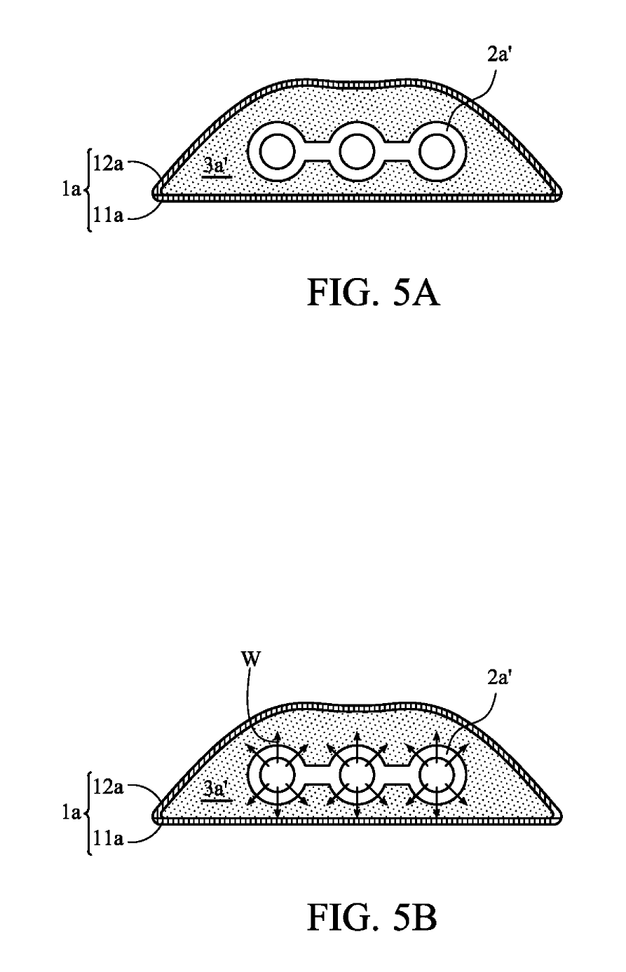

FIG. 5A shows another embodiment of the inner structure of the collecting device C1 of FIG. 3, and FIG. 5B is a schematic view showing the embodiment while collecting the fluid in FIG. 5A. FIG. 5A differs from FIG. 4A in that the embodiment comprises a first guiding element 2a′ and an absorbing element 3a′ which are integrally formed into one piece without the second guiding element 4a described above. In this embodiment, the first guiding element 2a′ and the absorbing element 3a′ are made of foam materials. Due to the first guiding element 2a′ provided with water permeability, the second guiding element 4a can be replaced by the first guiding element 2a′.

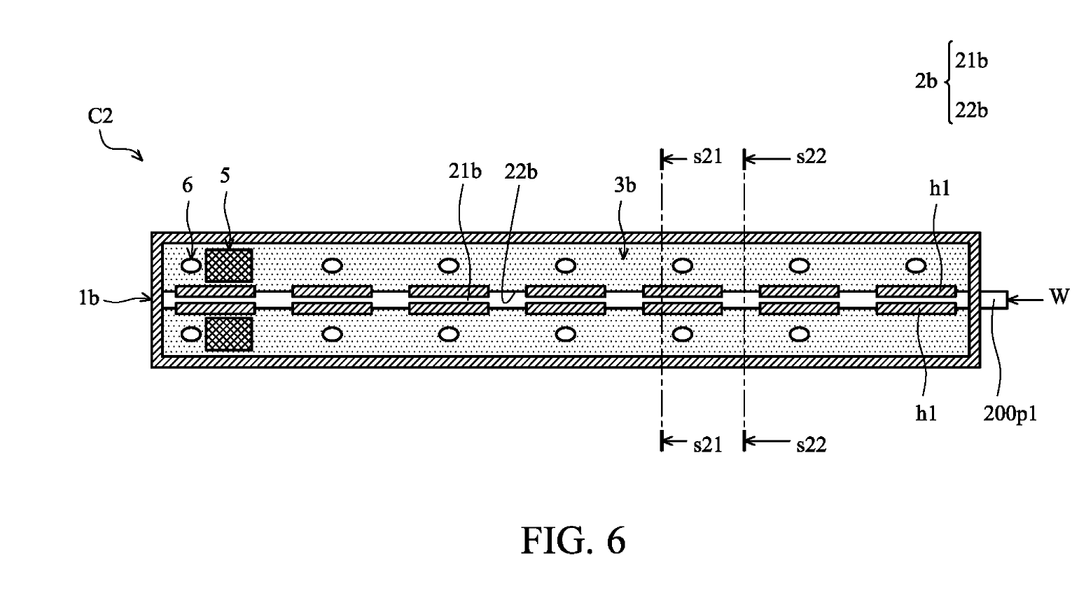

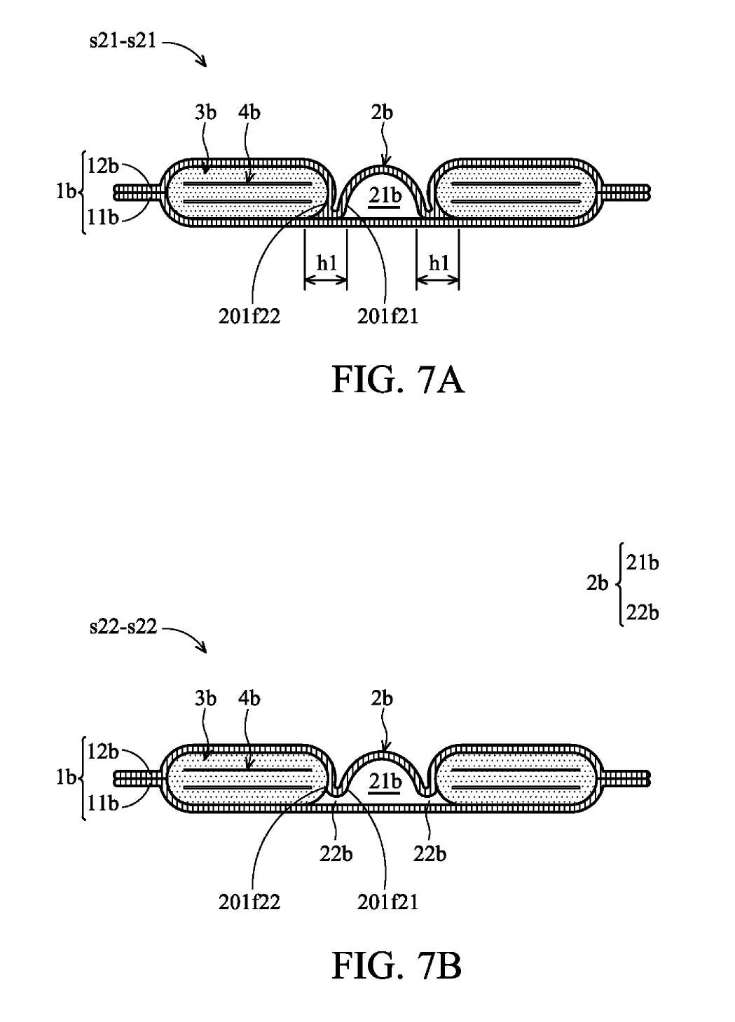

FIG. 6 is a schematic view of a collecting device C2 of a second embodiment, FIG. 7A is a sectional view showing the inner structure of the collecting device C2 along line s21-s21 in FIG. 6, and FIG. 7B is a sectional view showing the inner structure of the collecting device C2 along line s22-s22 in FIG. 6.

In FIGS. 6, 7A and 7B, the collecting device C2 comprises a base 1b, an absorbing element 3b, a guiding element 4b, and the air permeable region 5 and the display region 6.

The base 1b comprises a first layer 11b, a second layer 12b, a guiding region 2b and a connecting port 200p1. The guiding region 2b partially formed by the first and second layers 11b and 12b comprises a first channel 21b and a second channel 22b connected to the first channel 21b. The guiding region 2b further comprises a first wall surface 201f21 and a second wall surface 201f22. The first wall surface 201f21 forms the first channel 21b, and the second wall surface 201f22 disposed next to the absorbing element 3b is connected to the first wall surface 201f21 via the second channel 22b. That is, the guiding region 2b is a hollow structure formed between the first and second layers 11b and 12b. The connecting port 200p1 connected to the guiding region 2b is clamped between the first and second layers 11b and 12b of the base 1b. In this embodiment, the first and second layers 11b and 12b of the base 1b are formed by a water-resist separation layer or water-resist plastic membrane, the connecting port 200p1 is a pipe, and the guiding region 2b partially formed by the first and second layers 11b and 12b is formed by thermal bonding (e.g., ultrasonic method) or gluing. With thermal bonding, the first and second layers 11b and 12b of the base 1b further form a plurality of thermal-bonded regions h1 (shown in FIGS. 6 and 7B). Note that the profile of the first layer 11b of the base 1b is substantially flat.

The absorbing element 3b enclosed by the first and second layers 11b and 12b of the base 1b is disposed next to the guiding region 2b of the base 1b. That is, when the first and second layers 11b and 12b of the base 1b are thermally bonded with each other, the absorbing element 3b is also enclosed by the first and second layers 11b and 12b of the base 1b. In this embodiment, the absorbing element 3b is made of particulate water-absorbing resin material.

The guiding element 4b is disposed in the particulate absorbing element 3b, thereby guiding the waste liquid W adequately. In this embodiment, the guiding element 4b is a permeably water-guiding layer.

The air permeable region 5 is disposed on the first and second layers 11b and 12b of the base 1b to connect the interior of the base 1b to the exterior thereof, expelling air containing in the base 1b, the guiding region 2b and the absorbing element 3b to the exterior when the waste liquid W travels in the collecting device C2.

The display region 6 comprises wet-display dots disposed on the first and second layers 11b and 12b of the base 1b and connected to the absorbing element 3b to display the condition (e.g., saturated state of the absorbing element 3b), containing the waste liquid W. In this embodiment, the display region 6 made of water-discoloration material can be formed on the inner surface of the first and second layers 11b and 12b of the base 1b by printing.

In FIG. 6, when the waste liquid W driven by the negative pressure pump D2 is transmitted to the collecting devices C2 through an inlet 200p1, the waste liquid W traveling through the first and second channel 21b and 22b of the guiding region 2b is absorbed by the absorbing element 3b. Further, the air contained in the base 1b is adequately expelled to the exterior via the air permeable region 5, and the display region 6 shows the corresponding region of the absorbing element 3b being saturated or not by the change of the color thereof. Therefore, the absorption rate can be statistically estimated by the amount of the colored absorbing element 3b.

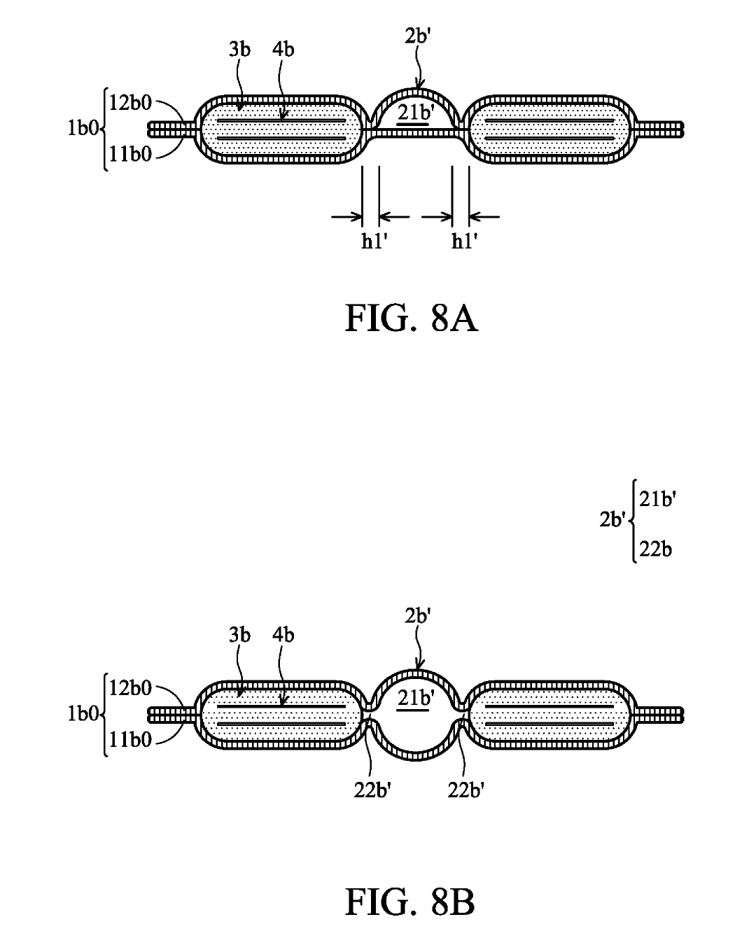

FIGS. 8A and 8B show another embodiment of the inner structures of the collecting device C2 of FIGS. 7A and 7B, respectively. The embodiment in FIGS. 8A and 8B differs from the collecting device C2 of FIGS. 7A and 7B in that an additional geometrical structure is provided. When comparing FIG. 8A to FIG. 7A and FIG. 7B to FIG. 8B, it is found that the locations of the first channel 21b′ of the guiding region 2b′ and thermal-bonded regions h1′ formed by the first and second layers 11b0 and 12b0 of the base 1b0 are locally uplifted as shown in FIG. 8A. Further, the section of the first channel 21b′ of the guiding region 2b′ is substantially formed as a circular shape and the locations of the second channels 22b′ are substantially located at the middle of the first channel 21b′ as shown in FIG. 8A. Additionally, the profile of the first layer 11b0 of the base 1b0 is curved and uneven, which is different from the flat first layer 11b of the base 1b shown in FIGS. 7A and 7B. In FIG. 8B, for example, the profile of the first and second layers 11b0 and 12b0 of the base 1b0 are horizontally symmetrical.

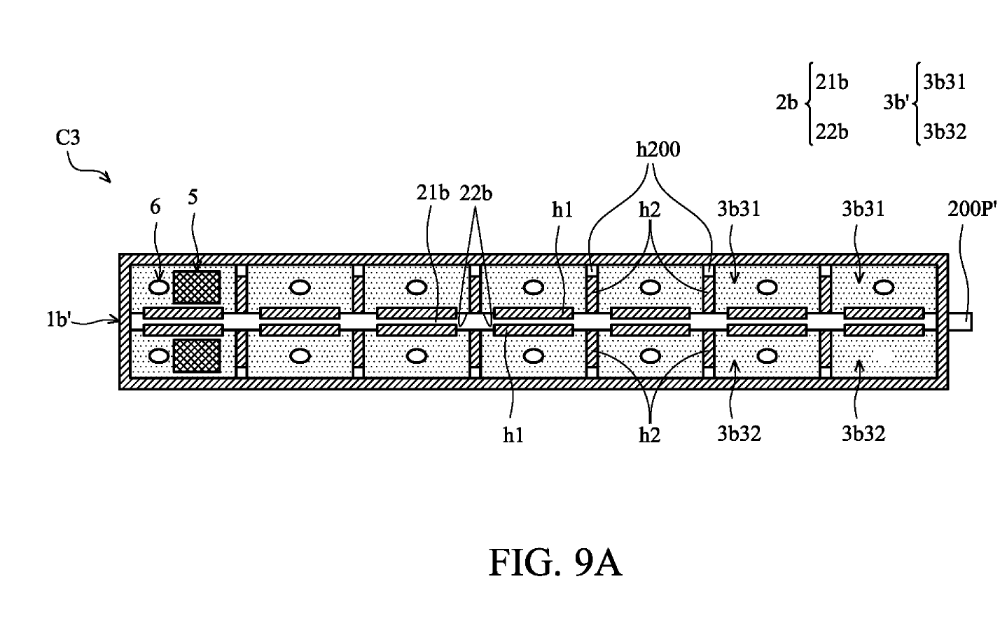

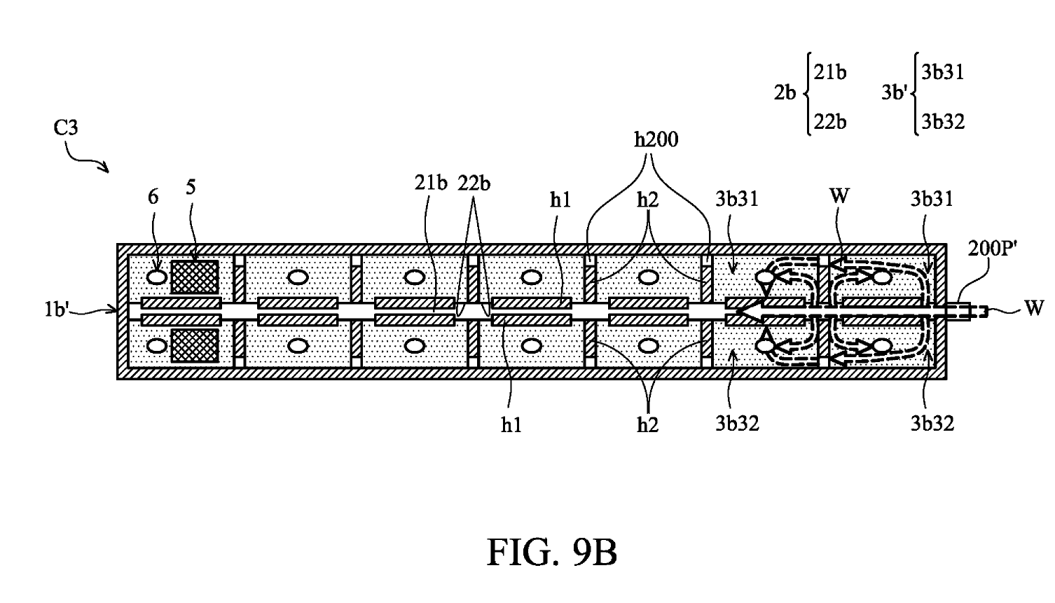

FIG. 9A is a schematic view of a collecting device C3 of a third embodiment, and FIG. 9B is a schematic view of the collecting device C3 while collecting the waste liquid W in FIG. 9A.

The collecting device C3 of FIG. 9A differs from the collecting device C2 of FIG. 6 in that the base 1b′ is further provided with a plurality of partitioned regions h2, thereby dividing the absorbing element 3b′ into a plurality of partial absorbing regions 3b31 and 3b32. The second channel 22b of the guiding region 2b is simultaneously connected to the partial absorbing regions 3b31 and 3b32 of the absorbing element 3b′. A plurality of channels h200 is formed between the partitioned regions h2 and the base 1b′. In this embodiment, the partitioned regions h2 are thermal bonding regions formed on the base 1b′ by thermal bonding (e.g., ultrasonic method).

In FIG. 9B, when the waste liquid W driven by the negative pressure pump D2 is transmitted to the collecting devices C3 through the connecting port 200p1, the waste liquid W traveling through the first and second channel 21b and 22b of the guiding region 2b is absorbed by the partial absorbing regions 3b3l and 3b32 of the absorbing element 3b′, and the partial absorbing regions 3b31 and 3b32 of the absorbing element 3b′ are balanced from each other by the connections of the channels h200. Further, the air contained in the base 1b′ is adequately expelled to the exterior via the air permeable region 5, and the display region 6 shows the corresponding partial absorbing regions 3b31 and 3b32 of the absorbing element 3b′ being saturated or not by the change of the color thereof. Therefore, the absorption rate can be statistically estimated by the amount of the colored absorbing element 3b′.

With the design of the partitioned regions and channels connected therebetween, the waste liquid W can arbitrarily diffuse to the partitioned regions, and thus the waste liquid W is precisely absorbed by the partial absorbing regions 3b31 and 3b32 of the absorbing element 3b′ while regularly diffusing. Further, with adequate installation of the display region 6 on the partial absorbing regions 3b31 and 3b32 of the absorbing element 3b′, the absorption condition of the collecting device can be precisely controlled according to the amount of the colored absorbing element 3b′.

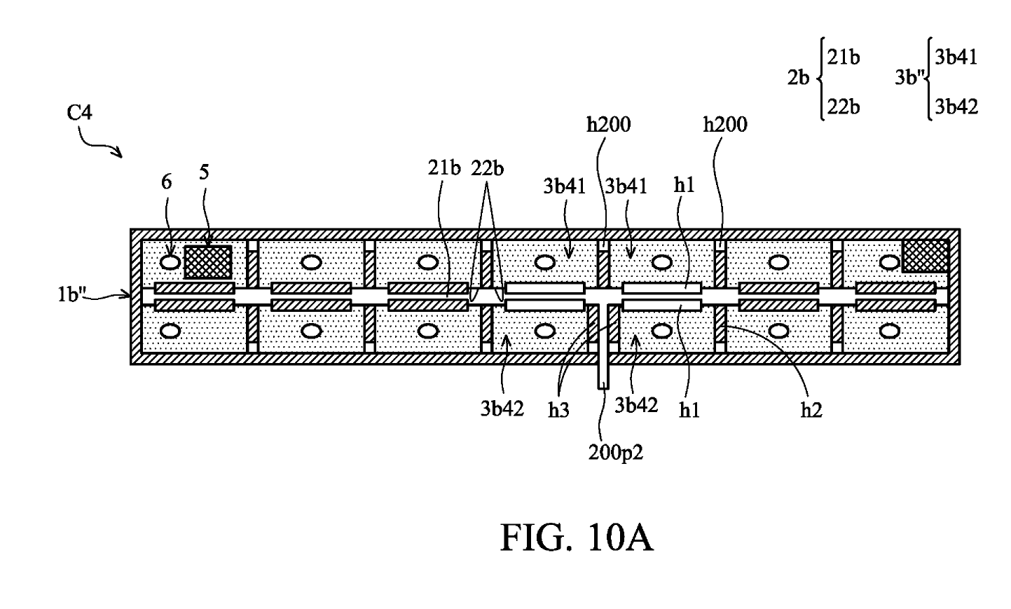

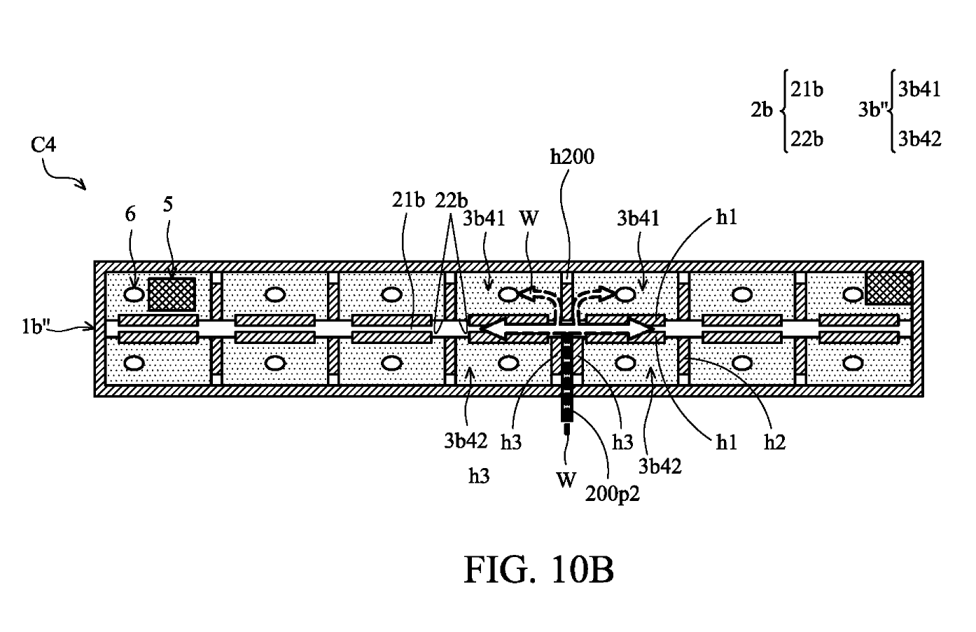

FIG. 10A is a schematic view of a collecting device C4 of a fourth embodiment, and FIG. 10B is a schematic view of the collecting device C4 while collecting the waste liquid W in FIG. 10A.

In FIG. 10A, compared to the collecting device C3 of the third embodiment, the base 1b″ of the collecting device C4 is divided into a plurality of partial absorbing regions 3b41 and 3b42, a connecting port 200p2 substantially disposed at the middle of the base 1b″ is connected to the first channel 21b of the guiding region 2b, two partitioned regions h3 are respectively located at both sides of the connecting port 200p2 to enforce the structure thereof, and two air permeable regions 5 are respectively disposed at two end portion of the base 1b″.

In FIG. 10B, when the waste liquid W driven by the negative pressure pump D2 is transmitted to the collecting devices C4 through the connecting port 200p2, the waste liquid W traveling through the first and second channel 21b and 22b of the first guiding element 2b is absorbed by the partial absorbing regions 3b41 and 3b42 of the absorbing element 3b″. Further, the air contained in the base 1b″ is adequately expelled to the exterior via the air permeable region 5, and the display region 6 shows the corresponding region of the absorbing element 3b″ being saturated or not by the change of the color thereof. Therefore, the absorption rate can be statistically estimated by the amount of the colored absorbing element 3b″.

While the invention has been described by way of example and in terms of several embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A collecting device utilized to collect a fluid, comprising:
a base;
a first guiding element enclosed by the base, comprising a first channel and a second channel connected to the first channel; and
an absorbing element disposed between the base and the first guiding element, absorbing the fluid guided by the first and second channels of the first guiding element.

2. The collecting device as claimed in claim 1, wherein the first guiding element further comprises an inner surface and an outer surface, the inner surface forms the first channel, and the outer surface disposed next to the absorbing element is connected to the inner surface via the second channel.

3. The collecting device as claimed in claim 1, wherein the first guiding element comprises a hollow structure.

4. The collecting device as claimed in claim 1, wherein the base comprises a first layer and a second layer, and the first guiding element is a hollow structure formed between the first and second layers of the base.

5. The collecting device as claimed in claim 1, wherein the first guiding element comprises silicone, rubber or plastic material.

6. The collecting device as claimed in claim 1 further comprising a second guiding element disposed between the first guiding element and the absorbing element, wherein the fluid guided by the first and second channels of the first guiding element and the second guiding element is absorbed by the absorbing element.

7. The collecting device as claimed in claim 6, wherein the second guiding element comprises a water-guiding layer disposed between the second channel of the first guiding element and the absorbing element.

8. The collecting device as claimed in claim 1, wherein the absorbing element comprises particulate water-absorbing resin material, hydrophilic plastic foam or water-absorbing resin foam material.

9. The collecting device as claimed in claim 1, wherein the first guiding element and the absorbing element are integrally or separably formed.

10. The collecting device as claimed in claim 9, wherein the first guiding element and the absorbing element comprises foam material.

11. The collecting device as claimed in claim 1 further comprising an air permeable region disposed on the base and utilized to connect the interior of the base to exterior.

12. The collecting device as claimed in claim 1 further comprising a display region disposed on the base and connected to the absorbing element to display the condition of the absorbing element containing the fluid.

13. The collecting device as claimed in claim 1, wherein the base comprises a water-resist separation layer or water-resist plastic membrane.

14. A collecting device utilized to collect a fluid, comprising:
a base comprising a first layer, a second layer, and a guiding region formed by the first and second layers and comprising a first channel and a second channel connected to the first channel; and
an absorbing element enclosed by the first and second layers of the base disposed next to the guiding region of the base, absorbing the fluid guided by the first and second channels of the guiding region of the base.

15. The collecting device as claimed in claim 14, wherein the guiding region of the base further comprises a first wall surface and a second wall surface, the first wall surface forms the first channel, and the second wall surface disposed next to the absorbing element is connected to the first wall surface via the second channel.

16. The collecting device as claimed in claim 14, wherein the guiding region of the base is a hollow structure formed between the first and second layers of the base.

17. The collecting device as claimed in claim 16, wherein the hollow structure formed between the first and second layers of the base is formed by thermal bonding or gluing.

18. The collecting device as claimed in claim 14 further comprising a guiding element disposed in the absorbing element.

19. The collecting device as claimed in claim 18, wherein the guiding element comprises a water-guiding layer.

20. The collecting device as claimed in claim 14, wherein the absorbing element comprises particulate water-absorbing resin material, hydrophilic plastic foam or water-absorbing resin foam material.

21. The collecting device as claimed in claim 14 further comprising an air permeable region disposed on the first and second layers of the base and utilized to connect the interior of the base to exterior.

22. The collecting device as claimed in claim 14 further comprising a display region disposed on the first and second layers of the base and connected to the absorbing element to display the condition of the absorbing element containing the fluid.

23. The collecting device as claimed in claim 14, wherein the base comprises a water-resist separation layer or water-resist plastic membrane.

24. The collecting device as claimed in claim 14, wherein the first and second layers of the base further form a partitioned region utilized to divide the absorbing element into at least two partial absorbing regions, and the second channel of the guiding region is simultaneously connected to the at least two partial absorbing regions.

25. The collecting device as claimed in claim 24, wherein the partitioned region is formed by thermal bonding or gluing.

26. A processing system utilized to process and absorb a fluid discharging from a source, comprising:
an absorbing device absorbing the fluid discharging from the source;
a driving device connected to the absorbing device, utilized to move the fluid collected by the absorbing device; and
a collecting device connected to the driving device and utilized to collect a fluid, comprising:
a base comprising a first layer, a second layer, and a guiding region formed by the first and second layers and comprising a first channel and a second channel connected to the first channel; and
an absorbing element enclosed by the first and second layers of the base disposed next to the guiding region of the base, absorbing the fluid guided by the first and second channels of the guiding region of the base.

27. The processing system as claimed in claim 26, wherein the guiding region of the base of the collecting device further comprises a first wall surface and a second wall surface, the first wall surface forms the first channel, and the second wall surface disposed next to the absorbing element is connected to the first wall surface via the second channel.

28. The processing system as claimed in claim 26, wherein the guiding region of the base of the collecting device is a hollow structure formed between the first and second layers of the base of the collecting device.

29. The processing system as claimed in claim 28, wherein the hollow structure formed between the first and second layers of the base of the collecting device is formed by thermal bonding or gluing.

30. The processing system as claimed in claim 26, wherein the collecting device further comprises a guiding element disposed in the absorbing element.

31. The processing system as claimed in claim 30, wherein the guiding element comprises a water-guiding layer.

32. The processing system as claimed in claim 26, wherein the absorbing element of the collecting device comprises particulate water-absorbing resin material, hydrophilic plastic foam or water-absorbing resin foam material.

33. The processing system as claimed in claim 26, wherein the collecting device further comprises an air permeable region disposed on the first and second layers of the base and utilized to connect the interior of the base to exterior.

34. The processing system as claimed in claim 26, wherein the collecting device further comprises a display region disposed on the first and second layers of the base and connected to the absorbing element to display the condition of the absorbing element containing the fluid.

35. The processing system as claimed in claim 26, wherein the base of the collecting device comprises a water-resist separation layer or water-resist plastic membrane.

36. The processing system as claimed in claim 26, wherein the first and second layers of the base of the collecting device further form a partitioned region utilized to divide the absorbing element into at least two partial absorbing regions, and the second channel of the guiding region is simultaneously connected to the at least two partial absorbing regions.

37. The processing system as claimed in claim 36, wherein the partitioned region of the base of the collecting device is formed by thermal bonding.

38. The processing system as claimed in claim 26, wherein the driving device is disposed between the absorbing device and the collecting device.

39. The processing system as claimed in claim 38, wherein the driving device comprises a negative pressure pump.

40. The processing system as claimed in claim 26, wherein the source comprises a wound, and the fluid comprises waste liquid or blood.
